(19) United States
(12) Patent Application Publication
(10) Pub. No.: US 2017/0252083 A1
(43) Pub. Date: Sep. 7, 2017

(54) SCREW THREAD ADAPTERS TO INCREASE SCREW FIXATION STRENGTH IN BONE

(71) Applicant: ARTHREX, INC., Naples, FL (US)

(72) Inventors: Matthew PALMER, Cambridge, MA (US); Matthew FONTE, Concord, MA (US); Robert DEVANEY, Auburndale, MA (US)

(21) Appl. No.: 15/451,557

(22) Filed: Mar. 7, 2017

Related U.S. Application Data
(60) Provisional application No. 62/304,471, filed on Mar. 7, 2016.

(51) Int. Cl. A61B 17/86

(52) U.S. Cl. CPC A61B 17/8685; A61B 17/866; A61B 17/864; A61B 2017/681

(57) ABSTRACT

This disclosure provides novel fixation devices which can be used with currently commercially available screws to increase their holding capacity (e.g., resistance to axial pull-out and shear).

### CROSS-REFERENCE TO RELATED APPLICATIONS

This application claims priority to U.S. Provisional Application No. 62/304,471, which was filed on Mar. 7, 2016, the disclosure of which is incorporated herein by reference in its entirety.

### TECHNICAL FIELD

This disclosure relates to screw thread adapters for increasing a screw's resistance to shearing in a human or animal body in order to facilitate healing of diseased or damaged tissue. The present disclosure finds particular utility in the field of orthopedics and specifically for aiding in reducing fractures and maintaining compression between bone fragments. While the present disclosure has application throughout the body, its utility will be illustrated herein in the context of the repair of fractured or displaced bone tissue.

### BACKGROUND

In the field of orthopedic surgery, it is common to rejoin broken bones. The success of the surgical procedure often depends on the ability to reapproximate the fractured bones, the amount of compression achieved between the bone fragments, and the ability to sustain that compression over a period of time. If the surgeon is unable to bring the bone fragments into close contact, a gap will exist between the bone fragments and the bone tissue will need to fill that gap before complete healing can take place. Furthermore, gaps between bone fragments that are too large allow motion to occur between the bone fragments, thus disrupting the healing tissue and slowing the healing process. Optimal healing requires that the bone fragments be in close contact with each other, and for a compressive load to be applied and maintained between the bone fragments. Compressive strain between bone fragments has been found to accelerate the healing process in accordance with Wolf's Law.

Broken bones can be rejoined using screws. Screws are typically manufactured from either titanium or stainless steel alloys and may be lag-type or headless. Lag screws have a distal threaded region and an enlarged head. The head contacts the cortical bone surface and the action of the threaded region reduces the fracture and generates a compressive load.

While these devices are designed to bring the bone fragments into close contact and to generate a compressive load between the bone fragments, these devices do not always succeed in accomplishing this objective. Among other things, the bone may be of low bone mineral density and thus not be able to resist the shear force exerted on it by the screw threads. This may cause the screw to “strip” in the bone resulting in a lack of compression.

Thus, there exists a clinical need for screws that are able to maintain grip in soft bone while the fracture heals.

### SUMMARY

This disclosure details novel fixation devices which can be used with currently commercially available screws to increase their holding capacity (e.g., resistance to axial pull-out and shear).

Among other things, the present disclosure includes the provision and use of a novel thread adapter which is manufactured from a shape memory material (e.g., a material capable of exhibiting superelasticity and/or a temperature-induced shape change). The shape memory material may be a metal alloy (e.g., Nitinol) or a polymer (e.g., appropriately processed PEEK). The thread adapter is designed to distally interface with a screw's threaded region and proximally flare outward, thus increasing the surface area available to resist shear and pull-out.

In an embodiment, the thread adapter includes a cannulated Nitinol rod that has fingers cut circumferentially around the rod. The rod is internally threaded with the threads designed to match available screws. In the un-constrained state, the fingers are flared radially outward. When implanted through an appropriately sized hole, the fingers collapse to a near parallel state to allow them to pass through the hole, and then expand outward once inside the bone.

In another embodiment, the threaded region of the thread adapter is replaced with a helical channel cut through the wall of the cannulated rod. The helical channel is sized to accommodate the screw thread geometry. The helical channel sits on the minor diameter of the screw thread. It should be appreciated that the major diameter of the screw thread may be greater than the outer diameter of the cannulated rod, thus allowing the screw threads to still grip into the bone. Furthermore the helical channel can act like a spring to allow a single thread insert to mate with screws of varying thread pitch.

In yet another embodiment, the thread adapter is made from a single piece of Nitinol wire. The Nitinol wire is coiled into a distal helix and proximally it is formed into petals. The helix can be sized to engage with the screw's threads. The helical coil sits on the minor diameter of the screw thread. It should be appreciated that the major diameter of the screw thread may be greater than the outer diameter of helical coil, thus allowing the screw threads to still grip into the bone. Furthermore the helical wire coil can act like a spring to allow a single threaded insert to mate with screws of differing thread pitch.

### BRIEF DESCRIPTION OF THE DRAWINGS

The various features and advantages of this disclosure will become apparent to those skilled in the art from the following detailed description. The drawings that accompany the detailed description can be briefly described as follows.

FIG. 1 is a schematic view of a novel thread adapter. The thread adapter is shown in the un-constrained state with finger regions radially expanding outward.

FIG. 2 is a schematic view of a novel thread adapter. The thread adapter is shown in the constrained state with the finger regions deformed to a parallel state.

FIG. 3 is a schematic view showing the novel thread adapter threaded onto a screw.

FIG. 4 shows a cross-sectional view of the thread adapter on the screw.

FIG. 5 shows an alternative schematic of an exemplary thread adapter. The thread adapter has a coil cut into the distal end and barbs cut out of the proximal end. The thread adapter is shown in the constrained state with the barbs nearly parallel.

FIG. 6 shows the thread adapter of FIG. 5 with the barbs in the radially expanded state.

FIG. 7 shows the thread adapter of FIGS. 5 and 6 on a screw with the helical cutout mated with screw threads.

FIG. 8 is an alternative schematic of a novel thread adapter. The thread adapter is formed from a single piece of wire and has a distal helical coil and a proximal petals for resisting axial pull-out. The thread adapter is shown in the un-constrained state.

FIG. 9 shows the thread adapter of FIG. 8 mated with a compression screw.

### DETAILED DESCRIPTION

A thread adapter for a screw includes a distal region sized to accommodate the screw and a proximal region extending from the distal region. The proximal region is radially expandable between a constrained state and an unconstrained state. A surface area of the screw is increased when the proximal region flexes toward the unconstrained state in order to increase an axial pull-out resistance of the screw.

In a further embodiment, a thread adapter is made of a shape memory material.

In a further embodiment, a shape memory material includes Nitinol.

In a further embodiment, a distal region of a thread adapter includes a threaded portion adapted to mate with threads of a screw.

In a further embodiment, a distal region of a thread adapter includes a helical channel.

In a further embodiment, a helical channel of a thread adapter is adapted to accommodate screw threads of varying pitches.

In a further embodiment, a proximal region and a distal region together establish a body of a thread adapter.

In a further embodiment, a body of a thread adapter is formed from a single piece of Nitinol wire.

In a further embodiment, a distal region of a thread adapter includes a helical coil and a proximal region includes at least one petal.

In a further embodiment, a distal region of a thread adapter includes a threaded portion or a helical portion, and a proximal region includes a finger, a barb, or a petal.

A method includes positioning a thread adapter over a screw, and inserting the screw into a bone hole after positioning the thread adapter over the screw. During the inserting, a region of the thread adapter collapses to allow passage into the bone hole and then flares radially outwardly once inside the bone hole.

In a further embodiment, a thread adapter is formed out of a shape memory material.

In a further embodiment, a shape memory material includes Nitinol.

In a further embodiment, a screw is a compression screw.

In a further embodiment, positioning a thread adapter includes inserting threads of a screw through a cannulation of the thread adapter.

In a further embodiment, positioning a thread adapter includes engaging threads of a screw with a threaded portion of the thread adapter.

In a further embodiment, positioning a thread adapter includes inserting threads of a screw through a helical channel of the thread adapter.

In a further embodiment, a region of a thread adapter includes at least one finger that flares radially outwardly to increase a surface area of a screw and thereby increase an axial pull-out resistance of the screw within a bone hole.

In a further embodiment, a region of a thread adapter includes at least one barb that flares radially outwardly to increase a surface area of a screw and thereby increase an axial pull-out resistance of the screw within a bone hole.

In a further embodiment, a region of a thread adapter includes at least one petal that flares radially outwardly to increase a surface area of a screw and thereby increase an axial pull-out resistance of the screw within a bone hole.

Devices to increase a grip of a screw in bone are of clinical need. Screws often strip in soft bone because the shear force the screw threads generate is greater than the shear strength of the underlying bone. Increasing the surface area available will increase the area the screw is able to spread the shear force over, thus not exceeding the bone's shear strength. One way to do this is by increasing the depth of a screws thread. An alternative way to do this is by threading an adapter or insert over the screw and allowing the adapter to expand radially, thus increasing the surface area available to distribute the shear force.

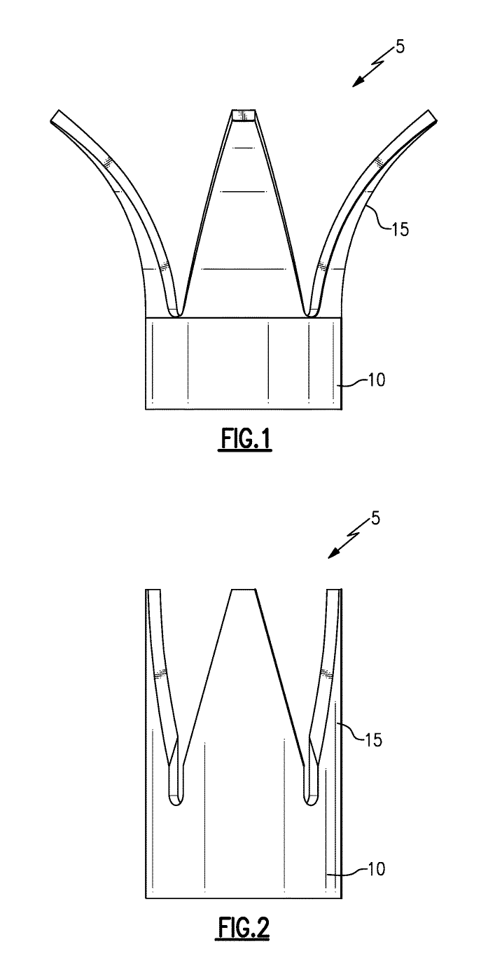

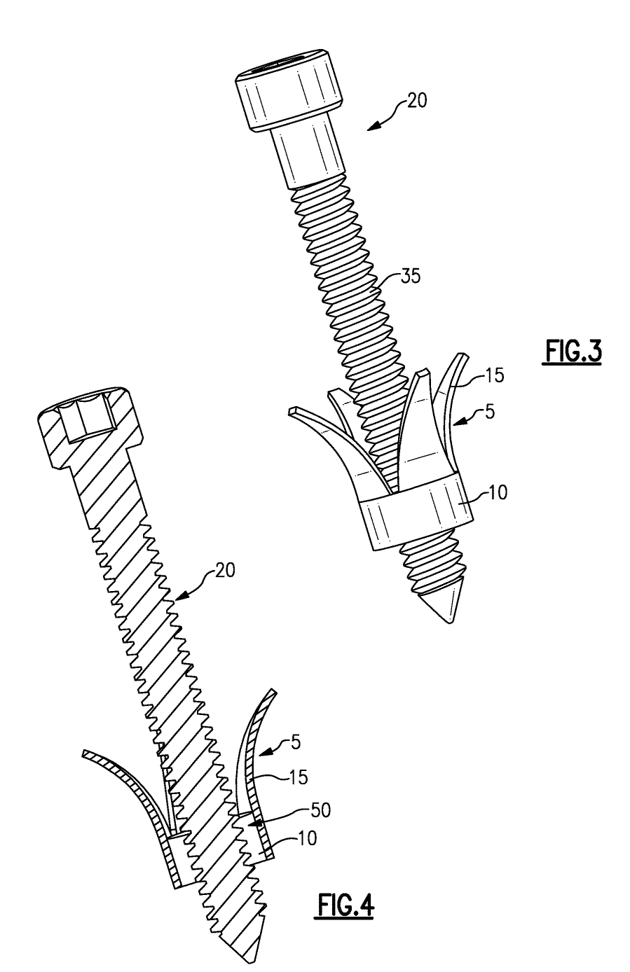

Referring first to FIGS. 1-4, there is shown a novel thread adapter 5. The thread adapter 5 has a base 10 (i.e., a distal region) and fingers 15 (i.e., a proximal region). In an unconstrained state, the fingers 15 are radially expanded. This increases the surface area available to a screw and thus increases resistance to axial pull-out. When the thread adapter 5 is being inserted through an appropriately sized drill hole, the fingers 15 can collapse to a near parallel configuration (see, for example, FIG. 2) to allow for insertion. Once inside the bone, the fingers 15 can radially expand.

As shown in FIGS. 3-4, the thread adapter 5 may be threaded onto a screw 20 prior to implantation. The thread adapter 5 may be received over screw threads 35 of the screw 20. The thread adapter 5 may be tapped or may have a threaded portion 50 that mates with the screw threads 35 of the screw 20 (see FIG. 4). Upon pressing the screw 20 into an appropriately drilled hole, the fingers 15 will collapse and subsequently radially flare open following implantation. Tightening the screw 20 will cause the threaded insert 5 to want to translate proximally toward the cortical surface. This will further radially expand the fingers.

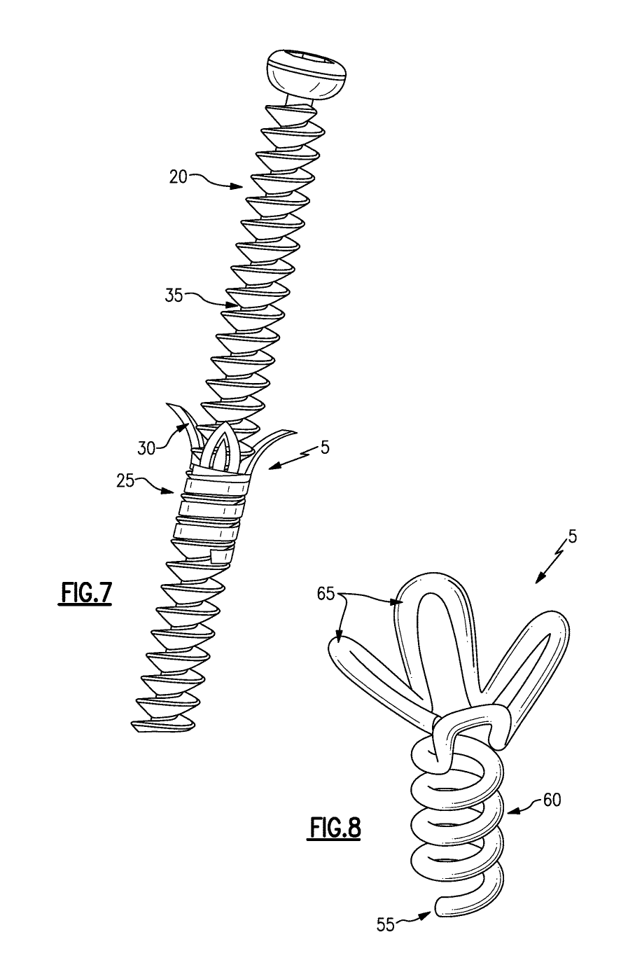

Referring now to FIGS. 5-7, there is shown an alternative embodiment of the thread adapter 5. In this embodiment, the thread adapter 5 has a distal helical channel 25 cut into its body. The helical channel 25 can accept screw threads 35 of a screw 20 (see FIG. 7). The helical channel 25 can act like a spring and can elongate or contract to accept screw threads 35 of varying pitches.

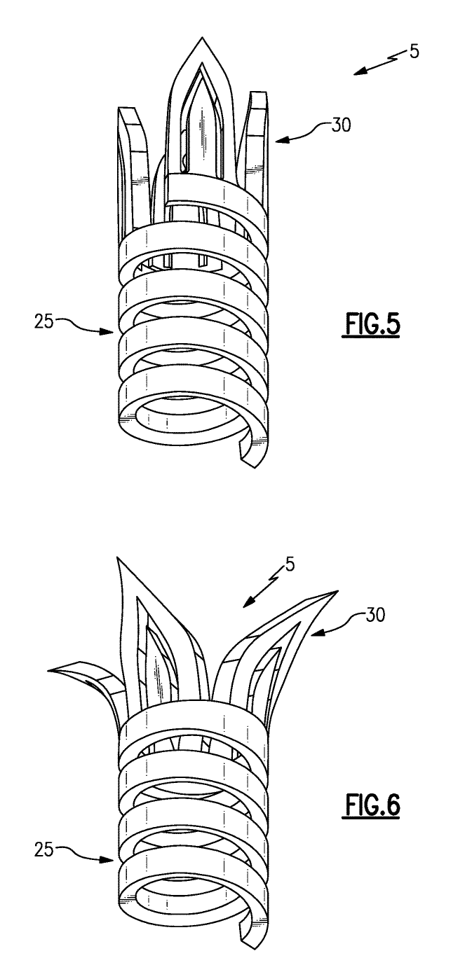

The thread adapter 5 of this embodiment includes barbs 30. The barbs 30 are shown in a constrained configuration in FIG. 5 and in an un-constrained configuration in FIG. 6. FIG. 7 shows the thread adapter 5 mating with the screw 20. As can be appreciated, the threads 35 sit inside the helical channel 25. The barbs 30 act to increase the surface area available to resist axial pull-out.

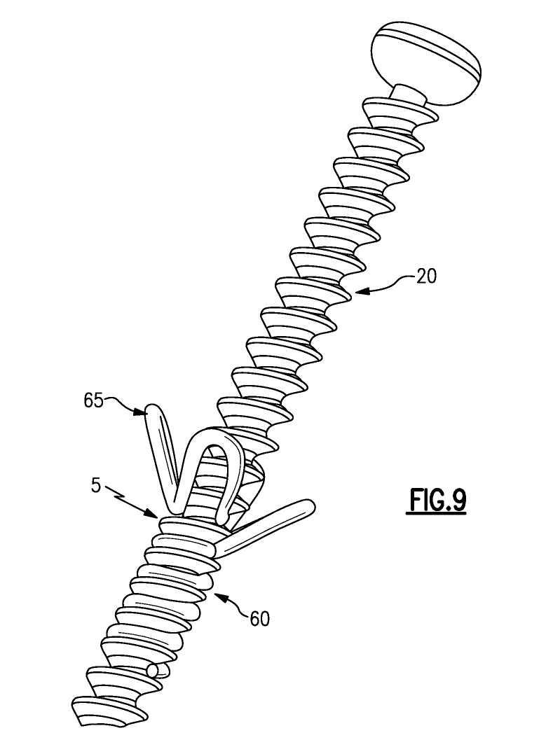

FIGS. 8 and 9 illustrate yet another embodiment of a thread adapter 5. In this embodiment, the thread adapter 5 is made of a single piece of wire 55. The wire 55 is shown as being round but could include other cross-sectional geometries. The wire 55 is distally wound into a helical coil 60 and is proximally shaped into one or more petals 65. The helical coil 60 can act like a spring and can elongate or contract to accept screw threads 35 of varying pitches. The petals 65 are shown in an un-constrained condition in FIG. 8 but could be bent to parallel during insertion into an appropriately sized hole. The petals 65 act to increase the surface area available to resist axial pull-out. FIG. 9 shows the thread adapter 5 of FIG. 8 engaged on a screw 20.

The foregoing description shall be interpreted as illustrative and not in any limiting sense. A worker of ordinary skill in the art would recognize that certain modifications could come within the scope of this disclosure. For these reasons, the following claims should be studied to determine the true scope and content of this disclosure.

What is claimed is:

1. A thread adapter for a screw, comprising:
a distal region sized to accommodate the screw; and
a proximal region extending from the distal region and being radially expandable between a constrained state and an unconstrained state, and wherein a surface area of the screw is increased when the proximal region flexes toward the unconstrained state in order to increase an axial pull-out resistance of the screw.

2. The thread adapter as recited in claim 1, wherein the thread adapter is made of a shape memory material.

3. The thread adapter as recited in claim 2, wherein the shape memory material comprises Nitinol.

4. The thread adapter as recited in claim 1, wherein the distal region includes a threaded portion adapted to mate with threads of the screw.

5. The thread adapter as recited in claim 1, wherein the distal region includes a helical channel.

6. The thread adapter as recited in claim 5, wherein the helical channel is adapted to accommodate screw threads of varying pitches.

7. The thread adapter as recited in claim 1, wherein the proximal region and the distal region together establish a body of the thread adapter.

8. The thread adapter as recited in claim 7, wherein the body is formed from a single piece of Nitinol wire.

9. The thread adapter as recited in claim 7, wherein the distal region includes a helical coil and the proximal region includes at least one petal.

10. The thread adapter as recited in claim 1, wherein the distal region includes a threaded portion or a helical portion, and the proximal region includes a finger, a barb, or a petal.

11. A method, comprising:
positioning a thread adapter over a screw;
inserting the screw into a bone hole after positioning the thread adapter over the screw; and
wherein during the inserting, a region of the thread adapter collapses to allow passage into the bone hole and then flares radially outwardly once inside the bone hole.

12. The method as recited in claim 11, wherein the thread adapter is formed out of a shape memory material.

13. The method as recited in claim 12, wherein the shape memory material comprises Nitinol.

14. The method as recited in claim 11, wherein the screw is a compression screw.

15. The method as recited in claim 11, wherein positioning the thread adapter includes inserting threads of the screw through a cannulation of the thread adapter.

16. The method as recited in claim 15, wherein positioning the thread adapter includes engaging threads of the screw with a threaded portion of the thread adapter.

17. The method as recited in claim 11, wherein positioning the thread adapter includes inserting threads of the screw through a helical channel of the thread adapter.

18. The method as recited in claim 11, wherein the region of the thread adapter includes at least one finger that flares radially outwardly to increase a surface area of the screw and thereby increase an axial pull-out resistance of the screw within the bone hole.

19. The method as recited in claim 11, wherein the region of the thread adapter includes at least one barb that flares radially outwardly to increase a surface area of the screw and thereby increase an axial pull-out resistance of the screw within the bone hole.

20. The method as recited in claim 11, wherein the region of the thread adapter includes at least one petal that flares radially outwardly to increase a surface area of the screw and thereby increase an axial pull-out resistance of the screw within the bone hole.
